(19) United States
(12) Patent Application Publication
(10) Pub. No.: US 2025/0067665 A1
(43) Pub. Date: Feb. 27, 2025

(54) COLD FILTER PLUGGING POINT PREDICTION USING SPECTROSCOPY

(71) Applicant: EXXONMOBIL TECHNOLOGY AND ENGINEERING COMPANY, Spring, TX (US)

(72) Inventors: Bryan T. BOWIE, Spring, TX (US); Jonathan L. SIMS, Hampshire (GB)

(73) Assignee: EXXONMOBIL TECHNOLOGY AND ENGINEERING COMPANY, Spring, TX (US)

(21) Appl. No.: 18/804,749

(22) Filed: Aug. 14, 2024

Related U.S. Application Data
(60) Provisional application No. 63/578,051, filed on Aug. 22, 2023.

(51) Int. Cl. G01N 21/3577; G01N 21/35; G01N 21/65; G01N 25/12; G01N 33/28

(52) U.S. Cl. CPC G01N 21/3577; G01N 21/65; G01N 2021/3595; G01N 25/12; G01N 33/2811

(57) ABSTRACT

Methods including: measuring a mid and/or near Fourier-transform infrared (FTIR) and/or Raman spectrum of a hydrocarbon fluid; determining an estimated cold filter plugging point (CFPP) for the hydrocarbon fluid using a vibrational spectroscopy-CFPP correlation model; and treating the hydrocarbon fluid to cause a change in the estimated CFPP. In some cases, the treating of the hydrocarbon fluid comprises: adding a flow improver to the hydrocarbon fluid.

### FIELD OF INVENTION

The present disclosure relates to quality control of hydrocarbon fluids using spectroscopy to predict a cold filter plugging point of said hydrocarbon fluid.

### BACKGROUND

Waxy molecules in distillate streams cause challenges in processing the molecules through hydrofiner units, and in the use of the fuels in vehicles at cold temperatures. The challenges stem from waxy molecules coalescing and impeding flow through filters. Manufacturers use a cold filter plugging point (CFPP) (the lowest temperature at which a given volume of diesel type of fuel still passes through a standardized filtration device in a specified time when cooled) as a measure of the amount of waxy molecules in the distillate or related streams.

The wax coalescing phenomenon can be circumvented by the addition of a flow improver. The flow improver disrupts the large surface area crystalline formation of wax, forcing smaller surface area crystals to be more favorable. However, the crystal types capable of being formed depend on the amount and the carbon number distribution of waxes present. Thus, determining the type of flow improver and amount required to treat the distillate stream or combination of streams can be challenging without knowing the carbon number distribution and quantity of wax by an analytical technique. However, the CFPP approach is more quantitative and reactionary. That is, the final distillate or fuel stream is analyzed with CFPP, then flow improvers may be used to adjust the CFPP to a desired value.

A newer method for analyzing wax uses a proprietary gas chromatography method to measure the carbon number distribution of the wax over a range of temperatures to determine physical properties of the stream including the cloud point (CP) and CFPP. The amount of precipitated wax at −30° C. is an indicator of the CP and the carbon number distribution of the wax helps with determining the CFPP. This data aids in the determination of the type and amount of flow improver to use. However, this method too is still a reactionary method because samples need to be sent to the vendor for evaluation to determine the flow improver type and amount, which can take days or longer and relies on testing capacity in the vendor laboratories. A measurement technique that allows for closer to real-time analysis of wax composition and concentration would be useful.

### SUMMARY OF INVENTION

Methods comprising: measuring a mid and/or near Fourier-transform infrared (FTIR) and/or Raman spectrum of a hydrocarbon fluid; determining an estimated cold filter plugging point (CFPP) for the hydrocarbon fluid using a vibrational spectroscopy-CFPP correlation model; and treating the hydrocarbon fluid to cause a change in the estimated CFPP.

Methods comprising: distilling a hydrocarbon feed into a plurality of cuts including a first hydrocarbon fluid cut; measuring a mid and/or near Fourier-transform infrared (FTIR) and/or Raman spectrum of the first hydrocarbon fluid cut; determining an estimated cold filter plugging point (CFPP) for the first hydrocarbon fluid cut using a vibrational spectroscopy-CFPP correlation model; and (a) treating the first hydrocarbon fluid cut to cause a change in the estimated CFPP, (b) changing a process parameter of the distilling of the hydrocarbon feed, or (c) performing both (a) and (b).

Machine-readable storage media having stored thereon a computer program for determining an estimated cold filter plugging point (CFPP) for a hydrocarbon fluid, the computer program comprising a routine set of instructions for causing the machine to perform the steps of: measuring a mid and/or near Fourier-transform infrared (FTIR) and/or Raman spectrum of a hydrocarbon fluid; and determining an estimated cold filter plugging point (CFPP) for the hydrocarbon fluid using a vibrational spectroscopy-CFPP correlation model.

These and other features and attributes of the disclosed methods and systems of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings. The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates a flow diagram related to a distillation and blending process where mid and/or near Fourier-transform infrared (FTIR) spectrophotometer and/or a Raman spectrometer (vibrational spectroscopy) is integrated according to at least some embodiments of the present disclosure.

FIG. 2 illustrates one example of a computer system 200 that can be employed to execute one or more embodiments of the present disclosure.

FIG. 3 is a representative parity plot for the validation of the −30° C. wax model. Similar plots can be generated for the other temperatures.

FIG. 4 is a plot of the least squares parity of the measured CFPP regressed against the predicted CFPP determined.

### DETAILED DESCRIPTION

The present disclosure relates to quality control of hydrocarbon fluids using spectroscopy to predict a cold filter plugging point of said hydrocarbon fluid. More specifically, spectra generated from a mid or near Fourier-transform infrared (FTIR) spectrophotometer or Raman spectrometer is used to estimate the amount and carbon number distribution of the wax in a hydrocarbon fluid and calculate or predict the CFPP of the hydrocarbon sample using a vibrational spectroscopy-CFPP correlation model.

Advantageously, collecting the FTIR spectrum and estimating the CFPP and/or wax concentration for a hydrocarbon sample according to the methods and systems described herein takes significantly less time (e.g., on the order of minutes or less) compared to gas chromatography and filter plugging analyses, which can take hours to days depending upon whether samples need to be shipped. Because the methods and system described provide closer to a real-time CFPP estimation, the CFPP can be used in more locations within the distillation and blending processes. This provides operators with more versatility in how to achieve a hydrocarbon fluid with a desired CFPP. For example, operators can adjust operation conditions (e.g., in a distillation tower) or additive and blending parameters (e.g., to blend to a desired CFPP or add a flow promoter to achieve a desired CFPP) on-the-fly to achieve the desired hydrocarbon fluid composition. This may improve efficiencies and lower costs associated with producing hydrocarbon fluids like distillates and fuels.

The methods and systems of the present disclosure for estimating the CFPP may be used, for example, in conjunction with a hydrocarbon fluid that includes hydrocarbons with a distillation point between 100° C. and 500° C. at 90 wt % or greater based on a total weight of the hydrocarbons in the hydrocarbon fluid. Examples of hydrocarbon fluids may include, but are not limited to, distillates, gasoline oils, heating oils, naphtha, diesel oils (e.g., a low sulfur diesel fuel, an ultra-low sulfur diesel fuel, a marine diesel fuel, an automotive diesel fuel), jet oils, and the like, and any blend thereof. As used herein, “oils” (e.g., gasoline oils, diesel oils, and the like) encompass feedstocks used to produce fuels and blended fuels (e.g., gasoline fuels, diesel fuels, and the like).

The hydrocarbon fluid may include n-alkanes and other hydrocarbon species that precipitate as wax as the hydrocarbon fluid cools. Said wax may plug filters and cause issues during transport of the hydrocarbon fluid or use of the hydrocarbon fluid in or as fuel. The CFPP provides an indication of the tendency for a hydrocarbon fluid to cause said plugging.

The methods and systems of the present disclosure may be used for estimating the CFPP of a hydrocarbon fluid using a vibrational spectroscopy-CFPP correlation model. Further, the systems and method of the present disclosure may include deriving a vibrational spectroscopy-CFPP correlation model for such use.

A vibrational spectroscopy-CFPP correlation model may be derived by measuring the vibrational spectra and measuring the CFPP for a plurality of samples. Then, one or more portions of the FTIR spectra may be correlated to the measured CFPP for each sample (e.g., measured using filter plugging methods and/or gas chromatography methods). Once a sufficient number of samples are analyzed, a correlation between the spectral analysis (e.g., intensity values at specific wavenumbers and/or integrated signal intensity over a range of wavenumbers) and the measured CFPP can be derived (or regressed) and validated.

The vibrational spectra for samples may, for example, be measured from an absolute or Raman spectrum shifted 200 cm⁻¹ to 7800 cm⁻¹ or a subrange thereof. The spectral data used in deriving (or regressing) and applying the vibrational spectroscopy-CFPP correlation model may be the intensity value at one or more specific wavenumbers and/or the integrated signal intensity over one or more ranges of wavenumbers. Optimization of wavelength/variable selection can done using commercial statistical packages or practices described in ASTM D8321 and/or E1655. An example of FTIR or Raman spectral regions that may preferably be used in the correlation may be one or more of 2600 cm⁻¹ to 3200 cm⁻¹, 800 cm⁻¹ to 2100 cm⁻¹, and 3500 cm⁻¹ to 4800 cm⁻¹, which again may be used as the ranges, subsets thereof may be used as ranges, or individual wavenumbers within the foregoing ranges may be used in deriving (or regressing) and applying the vibrational spectroscopy-CFPP correlation model of the present disclosure.

The measured CFPP may be measured using filter plugging methods such as ASTM D6371 and/or gas chromatography methods and/or Centrifugation-Gas Chromatography methods (Liu, H.; Duan, J.; Li, J.; Yan, H.; Wang, J.; Lin, K.; Guan, L.; Li, C. “Experimental Measurements of Wax Precipitation Using a Modified Method of Simultaneous Centrifugation and High Temperature Gas Chromatography,” Energies 2021, 14, 7035). An example filter plugging method is described in Journal of the Institute of Petroleum, 52 (1966), 173-185 and ASTM D6371.

Since the amount of wax that precipitates from the hydrocarbon fluid can be temperature dependent, the CFPP measurements or amount of solid wax and/or the FTIR spectra may be collected at one or more temperatures. The CFPP and FTIR need not be at the same temperature to proceed with the correlation. For example, the vibrational spectra may be measured at a first temperature, and the CFPP may be measured at a plurality of temperatures that may or may not include the first temperature. Then, separate vibrational spectroscopy-CFPP correlation models may be derived (or regressed) for the vibrational spectra at the first temperature correlated to the CFPP at each of the plurality of temperatures.

The temperature for the vibrational spectra and/or CFPP measurements used in deriving (or regressing) the vibrational spectroscopy-CFPP correlation model may independently be about −50° C. to about 20° C. (or about −50° C. to about −10° C., or about −30° C. to about 10° C., or about 0° C. to about 20° C.).

Examples of regressions suitable for deriving the correlation may include, but are not limited to, linear regression, polynomial regression, logistic regression, quantile regression, ridge regression, lasso regression, elastic net regression, principal component regression (PCR), partial least squares (PLS) regression, Locally Weighted Regression (LWR), Support Vector Machines Regression (SVM-R), Multiple Linear Regression (MLR), Classical Least Squares (CLS), Least Squares Regression (LSR), Artificial Neural Networks (ANN), Deep Learning Artificial Neural Networks (ANNDL), and the like.

The correlation may be any suitable mathematical formula. Examples of mathematical formulas may include, but are not limited to, a linear formula, a quadratic formula, an exponential correlation, regression vector, regression matrix, and the like.

Validation may, for example, be achieved using ASTM D6122.

Methods and systems of the present disclosure may include measuring a vibrational spectrum of a hydrocarbon sample from a hydrocarbon fluid; estimating a CFPP for the hydrocarbon fluid using a vibrational spectroscopy-CFPP correlation model of the present disclosure; and treating the hydrocarbon fluid to cause change to the CFPP.

The hydrocarbon fluid may, for example, be a hydrocarbon feedstock, a hydrocarbon stream, or a hydrocarbon product related to a distillation and/or blending process. The FTIR spectrophotometer and/or Raman spectrometer may be integrated into the systems that store and/or process hydrocarbon fluids. For example, the FTIR spectrometer may be located in-line with the hydrocarbon fluid stream in a distillation and/or blending process. Said system may include a plurality of in-line FTIR spectrophotometers and/or Raman spectrometers to monitor a plurality of hydrocarbon fluid lines in a distillation and/or blending process. In another example, one or more FTIR spectrophotometers and/or Raman spectrometers may be separate from the hydrocarbon fluid (e.g., in an on-site lab) such that samples of multiple hydrocarbon fluids may be collected and analyzed on-site. While in-line spectrometers provide the closest to real-time information, an on-site spectrometer still provides significant time benefits over other methods (e.g., gas chromatography and filter plugging methods) used to measure CFPP.

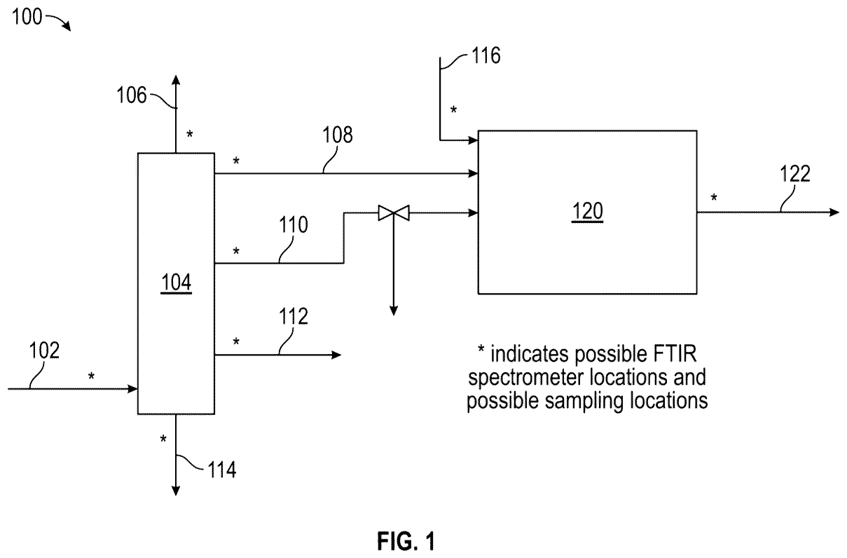

FIG. 1 illustrates a flow diagram related to a distillation and blending process 100. In the illustrated process, a first hydrocarbon feedstock stream 102 is introduced to a distillation tower 104 and separated into an overheads stream 106, a first cut stream 108, a second cut stream 110, a third cut stream 112, and a bottoms stream 114. The first cut stream 108, a portion of the second cut stream 110, and a second hydrocarbon feedstock stream 116 are introduced to a mixer 120 that blends the streams 108, 110, and 116 to produce a hydrocarbon product stream 122.

In FIG. 1, an asterisk is used to indicate possible locations for (a) placing an FTIR spectrophotometer and/or Raman spectrometer that is in-line with the relevant stream, (b) sampling the relevant stream for conducting measurements in an FTIR spectrophotometer and/or Raman spectrometer in a different location (e.g., in a lab) on-site, or (c) performing both (a) and (b). Said possible locations include, along the first hydrocarbon feedstock stream 102, along the overheads stream 106, along the first cut stream 108, along the second cut stream 110, along the third cut stream 112, along the bottoms stream 114, along the second hydrocarbon feedstock stream 116, and along the hydrocarbon product stream 122, where (a), (b), or (c) may be performed at one or more of the locations. For example, (b) may be performed at the location along the second hydrocarbon feedstock stream 116, (a) may be performed at the locations along the first cut stream 108 and the second cut stream 110, and (c) may be performed at the location along the hydrocarbon product stream 122.

One skilled in the art will recognize that FIG. 1 is a nonlimiting illustration. Other distillation and blending processes are contemplated as well as distillation processes that do not have a downstream blending process and blending where blending processes do not have a downstream distillation process. Also, the FTIR spectrophotometer and/or Raman spectrometer can be used at-line, near-line, or from a lab unit depending on the resources of the refinery.

Measurement of FTIR and/or Raman spectrum and determination of the estimated CFPP using the vibrational spectroscopy-CFPP correlation model of the present disclosure may be performed as a single measurement and determination, as a series of measurements and determinations at set time intervals (e.g., once per minute, once per hour, once per day, or the like), or as a series of continuous measurements and determinations (e.g., performed at least once every 30 seconds).

After estimating the CFPP for the hydrocarbon fluid using the vibrational spectroscopy-CFPP correlation model of the present disclosure, the hydrocarbon fluid may be treated to adjust the CFPP. The treatment may include adding a flow improver to the hydrocarbon fluid, adding another hydrocarbon fluid to the hydrocarbon fluid, and the like, or any combination thereof.

For example, a flow improver may be added to decrease the CFPP of the hydrocarbon fluid. The amount and composition of the flow improver may depend on the value of the CFPP, the composition of the hydrocarbon fluid, and the end-use of the hydrocarbon fluid.

Examples of flow improvers may include, but are not limited to, ethylene-vinyl ester copolymers (e.g., ethylene-vinyl acetate copolymers, ethylene-vinyl acetate-propionate copolymers), copolymers of ethylene and an ester of an unsaturated alcohol and a carboxylic acid, ethylene-vinyl chloride copolymers, the like, and any combination thereof. Examples of flow improvers are also described in U.S. Pat. Nos. 5,814,110 and 6,248,141, each of which is incorporated herein by reference.

The flow improver may be added to the hydrocarbon fluid in an amount of about 0.0005 wt % to about 1 wt % (or about 0.0005 wt % to about 0.01 wt %, or about 0.001 wt % to about 0.1 wt %, or about 0.01 wt % to about 1 wt %) based on the weight of the hydrocarbon fluid.

The selection of the composition and concentration of the flow improver may depend on the composition of the hydrocarbon fluid and/or the end-use of the hydrocarbon fluid.

In another treatment example, the CFPP of a first hydrocarbon fluid may be estimated using the vibrational spectroscopy-CFPP correlation model of the present disclosure. If the CFPP is too high, a second hydrocarbon fluid having a lower CFPP (which may be determined based on measurement or the vibrational spectroscopy-CFPP correlation model of the present disclosure) may be blended with the first hydrocarbon fluid to achieve a desired CFPP in the blend. Alternatively, if the CFPP is lower than the desired value, a second hydrocarbon fluid having a higher CFPP (which may be determined based on measurement or the vibrational spectroscopy-CFPP correlation model of the present disclosure) may be blended with the first hydrocarbon fluid in an amount so that the CFPP of the blend does not exceed the desired value.

The selection of the composition and amount of the second hydrocarbon fluid to be added to the first hydrocarbon fluid may depend on the composition of the first hydrocarbon fluid and/or the end-use of the blended hydrocarbon fluid product.

The treatments (e.g., addition of a flow improver and/or blending with another hydrocarbon fluid) may be done on-the-fly or in another step of the process. For example, the estimated CFPP of a first hydrocarbon fluid may be measured continuously (or with regular intervals) and continuously treated by adding a flow improver and/or blending with a second hydrocarbon fluid. The amount of flow improver and/or the amount of the second hydrocarbon fluid in the treatment may be changed in response to changes in the estimated CFPP.

In addition to treating the hydrocarbon fluid based on the estimated CFPP, the process used to produce the hydrocarbon fluid may be changed based on the CFPP. For example, in a distillation process, the CFPP of a cut stream may be measured continuously (or with regular intervals). When changes in the estimated CFPP are identified, one or more process parameters (e.g., a temperature in one or more zones of the distillation unit, a hydrocarbon feed rate, a hydrocarbon feed composition (or source), and the like) may be changed in such a way that causes the estimated CFPP to fall within a desired range for the cut stream. For example, the hydrocarbon feed may be a mixture from multiple sources where the ratio of the sources used to make the hydrocarbon feed for the distillation process may be adjusted in response to changes to the estimated CFPP.

The hydrocarbon product after treating the hydrocarbon fluid and/or changing a process parameter may, for example, be a fuel or a fluid used to blend with other hydrocarbon fluids and/or additives to produce a fuel (e.g., gasoline fuel, heating fuel, diesel fuel, jet fuel, and the like).

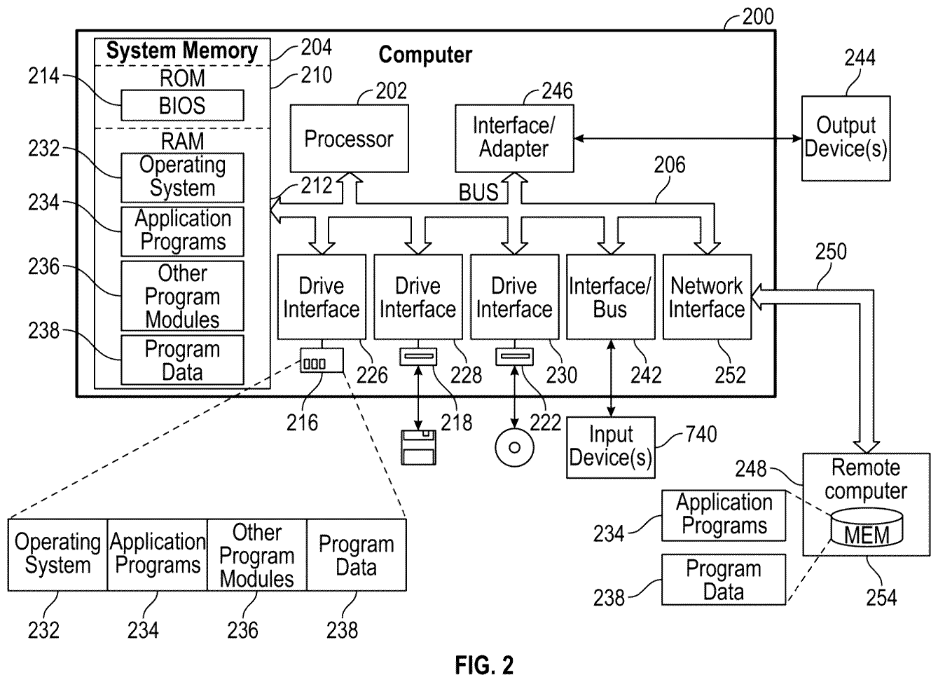

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 2. Furthermore, portions of the embodiments may be a computer program product on a computer-usable storage medium having computer readable program code on the medium. Any non-transitory, tangible storage media possessing structure may be utilized including, but not limited to, static and dynamic storage devices, hard disks, optical storage devices, and magnetic storage devices, but excludes any medium that is not eligible for patent protection under 35 U.S.C. § 101 (such as a propagating electrical or electromagnetic signals per sc). As an example and not by way of limitation, computer-readable storage media may include a semiconductor-based circuit or device or other Integrated Circuit (IC) (such, as for example, a field-programmable gate array (FPGA) or an Application Specific IC), a hard disk, a hard disk drive (HDD), a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a secure digital (SD) card, a SD drive, or another suitable computer-readable storage medium or a combination of two or more of these, where appropriate. A computer-readable, non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine set of instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods, and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

In this regard, FIG. 2 illustrates one example of a computer system 200 that can be employed to execute one or more embodiments of the present disclosure. Computer system 200 can be implemented on one or more general purpose networked computer systems, embedded computer systems, routers, switches, server devices, client devices, various intermediate devices/nodes, or standalone computer systems. Additionally, computer system 200 can be implemented on various mobile clients such as, for example, a personal digital assistant (PDA), laptop computer, pager, and the like, provided it includes sufficient processing capabilities.

Computer system 200 includes processing unit 202, system memory 204, and system bus 206 that couples various system components, including the system memory 204, to processing unit 202. Dual microprocessors and other multi-processor architectures also can be used as processing unit 202. System bus 206 may be any of several types of bus structure including a memory bus or memory controller, a peripheral bus, and a local bus using any of a variety of bus architectures. System memory 204 includes read only memory (ROM) 210 and random access memory (RAM) 212. A basic input/output system (BIOS) 214 can reside in ROM 210 containing the basic routines that help to transfer information among elements within computer system 200.

Computer system 200 can include a hard disk drive 216, magnetic disk drive 218, e.g., to read from or write to removable disk, and an optical disk drive 222, e.g., for reading CD-ROM disk or to read from or write to other optical media. Hard disk drive 216, magnetic disk drive 218, and optical disk drive 222 are connected to system bus 206 by a hard disk drive interface 226, a magnetic disk drive interface 228, and an optical drive interface 230, respectively. The drives and associated computer-readable media provide nonvolatile storage of data, data structures, and computer-executable instructions for computer system 200. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 210, including operating system 232, one or more application programs 234, other program modules 236, and program data 238. In some examples, the application programs 234 can include applications that derive (or regress) some or all of the vibrational spectroscopy-CFPP correlation model of the present disclosure and applications that implement the vibrational spectroscopy-CFPP correlation model of the present disclosure, and the program data 238 can include estimated CFPP and related recommendations regarding treatments to the hydrocarbon fluid (e.g., the addition of flow improvers and/or other hydrocarbon fluids for blending) to change the CFPP of the hydrocarbon fluid. The application programs 234 and program data 238 can include functions and methods programmed to derive (or regress) the vibrational spectroscopy-CFPP correlation model of the present disclosure, apply the vibrational spectroscopy-CFPP correlation model of the present disclosure, and perform an action in response to an estimated CFPP from the vibrational spectroscopy-CFPP correlation model of the present disclosure, such as shown and described herein.

A user may enter commands and information into computer system 200 through one or more input devices 740, such as a pointing device (e.g., a mouse, touch screen), keyboard, microphone, joystick, game pad, scanner, and the like. For instance, the user can employ input device 740 to edit or modify a desired value or range for the CFPP of a hydrocarbon fluid, instructions regarding the method or thresholds associated with deriving (or regressing) the vibrational spectroscopy-CFPP correlation model of the present disclosure, and instructions regarding the application (or use) of the vibrational spectroscopy-CFPP correlation model of the present disclosure. These and other input devices 740 are often connected to processing unit 202 through a corresponding port interface 242 that is coupled to the system bus, but may be connected by other interfaces, such as a parallel port, serial port, or universal serial bus (USB). One or more output devices 244 (e.g., display, a monitor, printer, projector, or other type of displaying device) is also connected to system bus 206 via interface 246, such as a video adapter.

Computer system 200 may operate in a networked environment using logical connections to one or more remote computers, such as remote computer 248. Remote computer 248 may be a workstation, computer system, router, peer device, or other common network node, and typically includes many or all of the elements described relative to computer system 200. The logical connections, schematically indicated at 250, can include a local area network (LAN) and/or a wide area network (WAN), or a combination of these, and can be in a cloud-type architecture, for example, configured as private clouds, public clouds, hybrid clouds, and multi-clouds. When used in a LAN networking environment, computer system 200 can be connected to the local network through a network interface or adapter 252. When used in a WAN networking environment, computer system 200 can include a modem, or can be connected to a communications server on the LAN. The modem, which may be internal or external, can be connected to system bus 206 via an appropriate port interface. In a networked environment, application programs 234 or program data 238 depicted relative to computer system 200, or portions thereof, may be stored in a remote memory storage device 254.

Unless otherwise indicated, all numbers expressing quantities of ingredients, properties such as carbon number, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

### ADDITIONAL EMBODIMENTS

Clause 1: A method comprising: measuring a mid and/or near Fourier-transform infrared (FTIR) and/or Raman spectrum of a hydrocarbon fluid; determining an estimated cold filter plugging point (CFPP) for the hydrocarbon fluid using a vibrational spectroscopy-CFPP correlation model; and treating the hydrocarbon fluid to cause a change in the estimated CFPP.

Clause 2: The method of Clause 1, wherein the treating of the hydrocarbon fluid comprises: adding a flow improver to the hydrocarbon fluid.

Clause 3: The method of any preceding Clause, wherein the hydrocarbon fluid is a first hydrocarbon fluid, and wherein the treating of the first hydrocarbon fluid comprises: adding a second hydrocarbon fluid to the hydrocarbon fluid.

Clause 4: The method of any preceding Clause further comprising: blending the first hydrocarbon fluid with at least one additional hydrocarbon fluid to produce a fuel.

Clause 5: The method of any preceding Clause, wherein the measuring of the vibrational spectrum is part of continuously measuring the vibrational spectrum of hydrocarbon fluid, and wherein the determining of the estimated CFPP is part of monitoring the estimated CFPP over time.

Clause 6: The method of any preceding Clause, wherein the CFPP is for a total wax concentration.

Clause 7: The method of any preceding Clause, wherein the CFPP is for a wax concentration at a temperature between about 50° C. to about 20° C.

Clause 8: The method of any preceding Clause, wherein the hydrocarbon fluid is a distillate.

Clause 9: The method of any preceding Clause, wherein the hydrocarbon fluid is a low sulfur diesel fuel or an ultra-low sulfur diesel fuel.

Clause 10: A method comprising: distilling a hydrocarbon feed into a plurality of cuts including a first hydrocarbon fluid cut; measuring a mid and/or near Fourier-transform infrared (FTIR) and/or Raman spectrum of the first hydrocarbon fluid cut; determining an estimated cold filter plugging point (CFPP) for the first hydrocarbon fluid cut using a vibrational spectroscopy-CFPP correlation model; and (a) treating the first hydrocarbon fluid cut to cause a change in the estimated CFPP, (b) changing a process parameter of the distilling of the hydrocarbon feed, or (c) performing both (a) and (b).

Clause 11: The method of Clause 10, wherein the plurality of cuts further includes a second hydrocarbon fluid cut, and wherein the treating of the first hydrocarbon fluid cut comprises blending at least a portion of the second hydrocarbon fluid cut into the first hydrocarbon fluid cut.

Clause 12: The method of Clause 10 or 11, wherein the process parameter includes one or more of: a temperature in one or more zones of a distillation unit, a hydrocarbon feed rate, a composition of the hydrocarbon feed, and any combination thereof.

Clause 13: The method of Clause 10, 11, or 12, wherein the treating of the first hydrocarbon fluid cut comprises: adding a flow improver to the first hydrocarbon fluid cut.

Clause 14: The method of Clause 10, 11, 12, or 13, wherein the measuring of the vibrational spectrum is part of continuously measuring the vibrational spectrum of first hydrocarbon fluid, and wherein the determining of the estimated CFPP is part of monitoring the estimated CFPP over time.

Clause 15: The method of Clause 10, 11, 12, 13, or 14, wherein the CFPP is for a total wax concentration.

Clause 16: The method of Clause 10, 11, 12, 13, 14, or 15, wherein the CFPP is for a wax concentration at a temperature between about 50° C. to about 20° C.

Clause 17: A machine-readable storage medium having stored thereon a computer program for determining an estimated cold filter plugging point (CFPP) for a hydrocarbon fluid, the computer program comprising a routine set of instructions for causing the machine to perform the steps of: measuring a mid and/or near Fourier-transform infrared (FTIR) and/or Raman spectrum of a hydrocarbon fluid; and determining an estimated cold filter plugging point (CFPP) for the hydrocarbon fluid using a vibrational spectroscopy-CFPP correlation model.

Clause 18: The machine-readable storage medium of Clause 17, wherein the measuring of the vibrational spectrum is part of continuously measuring the vibrational spectrum of hydrocarbon fluid, and wherein the determining of the estimated CFPP is part of monitoring the estimated CFPP over time.

Clause 19: The machine-readable storage medium of Clause 17 or 18, wherein the CFPP is for a total wax concentration.

Clause 20: The machine-readable storage medium of Clause 17, 18, or 19, wherein the CFPP is for a wax concentration at a temperature between about −50° C. to about 20° C.

To facilitate a better understanding of the embodiments of the present invention, the following examples of preferred or representative embodiments are given. In no way should the following examples be read to limit, or to define, the scope of the invention.

### EXAMPLES

383 samples of at least 12 different cuts of whole crude including neutral distillates and no diesel samples were used to develop a vibrational spectroscopy-CFPP correlation model. FTIR spectra and calorimetric (differential scanning calorimetry, DSC) data were gathered for the samples. The calorimetric data provided the concentration and distribution of the wax in the sample as a function of temperature. The FTIR and calorimetric data were regressed (using a partial least squares regression and optimized using techniques described in D8321 and D6122) to produce a vibrational spectroscopy-CFPP correlation model that predicted the total wax concentration and the concentration of wax that would contribute to the CFPP at −30° C., −18° C., and −9° C. Table 1 below shows the calibration range, number of calibration samples, root mean square error of calibration (RMSEC), and the equation for the property level dependent error. Table 2 shows the validation range, number of validation samples, root mean square error of prediction (RMSEP) and if it passed ASTM D6122-23 local validation criteria.

TABLE 1
Calibration

| | Wax | | |
|---|---|---|---|
| | Concentration | # | |
| | Calibration | Cali- | |
| | Range | bration | |
| Prediction | [Wt %] | Samples | Level Dependent Error |
| Total Wax | 0.3-66.7 | 246 | t(SEC) = 0.01907*X + 2.954 |
| −30° C. Wax | 0.1-66.2 | 223 | t(SEC) = 0.01851*X + 3.474 |
| −18° C. Wax | 0.1-60.5 | 217 | t(SEC) = 0.06472*X + 2.412 |
| −9° C. Wax | 0.2-63.8 | 189 | t(SEC) = 0.03174*X + 2.320 |

TABLE 2
Validation

| | Wax | | |
|---|---|---|---|
| | Concentration | | |
| | Validation Range | # Validation | ASTM D6122-21 |
| Validation | [Wt %] | Samples | (pass/fail) |
| Total Wax | 0.7-58.6 | 81 | Pass |
| −30° C. Wax | 0.2-62.5 | 125 | Pass |
| −18° C. Wax | 0.1-45.0 | 125 | Pass |
| −9° C. Wax | 0.2-59.6 | 63 | Pass |

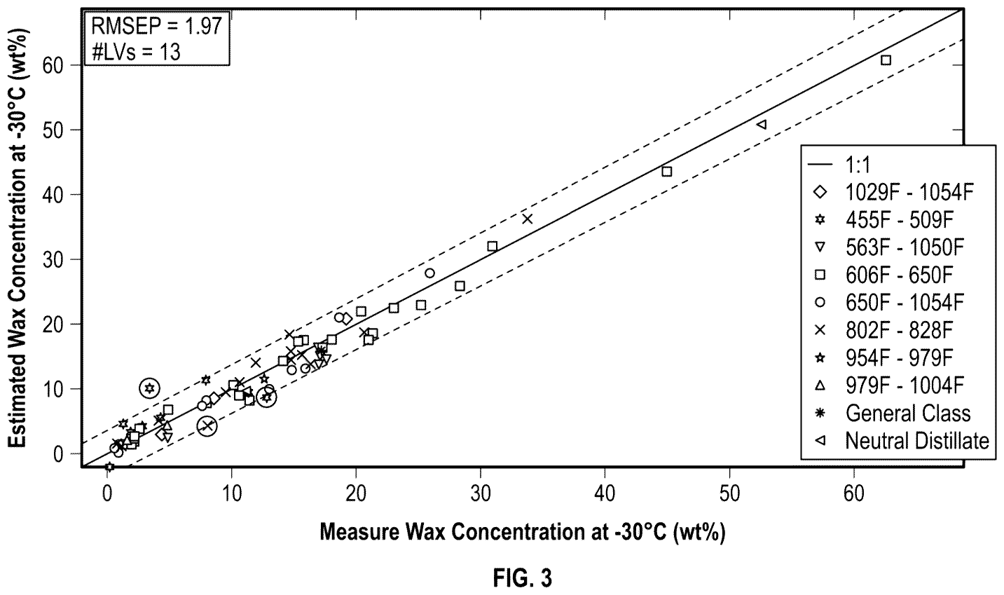

FIG. 3 is a representative parity plot for the validation of the −30° C. wax model. Similar plots can be generated for the other temperatures. In FIG. 3, the legend shows the different whole crude cuts used to validate the vibrational spectroscopy-CFPP correlation model. The dashed lines are the 95% confidence level. The circled data points fell outside the 95% confidence interval. As many as 6 of the 124 validation samples could fall outside the 95% confidence level in order to pass ASTM D6122-21 local validation criteria.

Using the validated vibrational spectroscopy-CFPP correlation model at −30° C., diesel samples were collected and analyzed (1) via FTIR while on-site and (2) via gas chromatography by a vendor, which required shipping samples to said vendor. Generally, the data between the vibrational spectroscopy-CFPP correlation model at −30° C. and gas chromatography results follow the same trends. Therefore, the vibrational spectroscopy-CFPP correlation model at −30° C. appears to be good enough to apply in real-time to avoid disruption and know when and how much flow improver to add to a formulation.

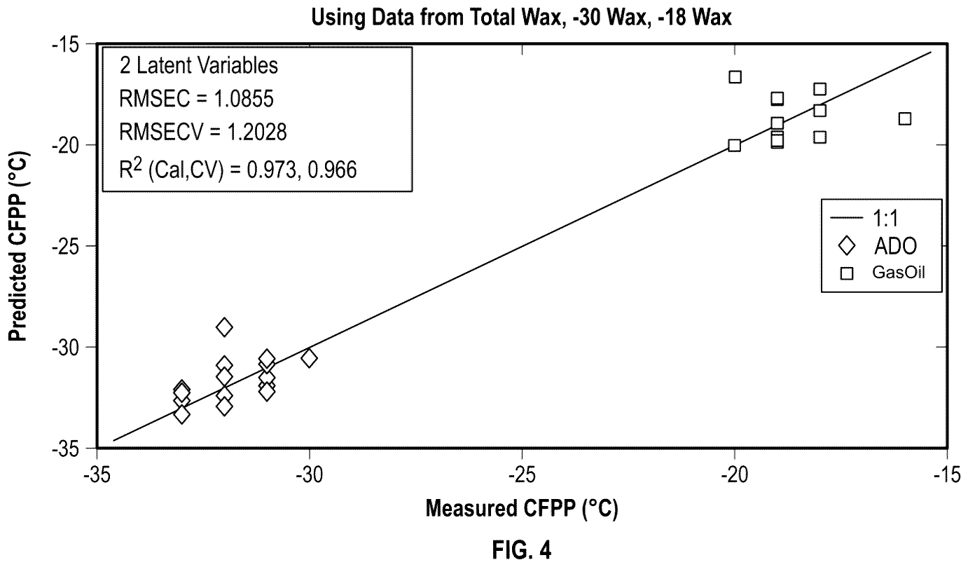

In another example of using validated vibrational spectroscopy-CFPP correlation models, several samples of automotive diesel oil (ADO) and marine gas oil were collected and analyzed (1) via FTIR while on-site (predicted CFPP) and (2) via a cloud point measurement to determine CFPP (measured CFPP), which required shipping samples to another site. FIG. 4 is a plot of the least squares parity of the measured CFPP regressed against the predicted CFPP determined. The data plotted in FIG. 5 includes lab CFPP and predicted CFPP values for total wax, wax at −30° C., and wax at −18° C. Again, the data is well correlated, which further validates the use of vibrational spectroscopy-CFPP correlation models for real-time to near real-time CFPP determination, which, in turn, may avoid disruptions, optimize output, and reduce cost from excessive flow improver in fuel and distillate formulations.

Therefore, the present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

What is claimed is:

1. A method comprising:
measuring a mid and/or near Fourier-transform infrared (FTIR) and/or Raman spectrum of a hydrocarbon fluid;
determining an estimated cold filter plugging point (CFPP) for the hydrocarbon fluid using a vibrational spectroscopy-CFPP correlation model; and
treating the hydrocarbon fluid to cause a change in the estimated CFPP.

2. The method of claim 1, wherein the treating of the hydrocarbon fluid comprises: adding a flow improver to the hydrocarbon fluid.

3. The method of claim 1, wherein the hydrocarbon fluid is a first hydrocarbon fluid, and wherein the treating of the first hydrocarbon fluid comprises: adding a second hydrocarbon fluid to the hydrocarbon fluid.

4. The method of claim 1 further comprising:
blending the first hydrocarbon fluid with at least one additional hydrocarbon fluid to produce a fuel.

5. The method of claim 1, wherein the measuring of the vibrational spectrum is part of continuously measuring the vibrational spectrum of hydrocarbon fluid, and wherein the determining of the estimated CFPP is part of monitoring the estimated CFPP over time.

6. The method of claim 1, wherein the CFPP is for a total wax concentration.

7. The method of claim 1, wherein the CFPP is for a wax concentration at a temperature between about −50° C. to about 20° C.

8. The method of claim 1, wherein the hydrocarbon fluid is a distillate.

9. The method of claim 1, wherein the hydrocarbon fluid is a low sulfur diesel fuel or an ultra-low sulfur diesel fuel.

10. A method comprising:
distilling a hydrocarbon feed into a plurality of cuts including a first hydrocarbon fluid cut;
measuring a mid and/or near Fourier-transform infrared (FTIR) and/or Raman spectrum of the first hydrocarbon fluid cut;
determining an estimated cold filter plugging point (CFPP) for the first hydrocarbon fluid cut using a vibrational spectroscopy-CFPP correlation model; and
(a) treating the first hydrocarbon fluid cut to cause a change in the estimated CFPP, (b) changing a process parameter of the distilling of the hydrocarbon feed, or (c) performing both (a) and (b).

11. The method of claim 10, wherein the plurality of cuts further includes a second hydrocarbon fluid cut, and wherein the treating of the first hydrocarbon fluid cut comprises blending at least a portion of the second hydrocarbon fluid cut into the first hydrocarbon fluid cut.

12. The method of claim 10, wherein the process parameter includes one or more of: a temperature in one or more zones of a distillation unit, a hydrocarbon feed rate, a composition of the hydrocarbon feed, and any combination thereof.

13. The method of claim 10, wherein the treating of the first hydrocarbon fluid cut comprises: adding a flow improver to the first hydrocarbon fluid cut.

14. The method of claim 10, wherein the measuring of the vibrational spectrum is part of continuously measuring the vibrational spectrum of first hydrocarbon fluid, and wherein the determining of the estimated CFPP is part of monitoring the estimated CFPP over time.

15. The method of claim 10, wherein the CFPP is for a total wax concentration.

16. The method of claim 10, wherein the CFPP is for a wax concentration at a temperature between about −50° C. to about 20° C.

17. A machine-readable storage medium having stored thereon a computer program for determining an estimated cold filter plugging point (CFPP) for a hydrocarbon fluid, the computer program comprising a routine set of instructions for causing the machine to perform the steps of:
measuring a mid and/or near Fourier-transform infrared (FTIR) and/or Raman spectrum of a hydrocarbon fluid; and
determining an estimated cold filter plugging point (CFPP) for the hydrocarbon fluid using a vibrational spectroscopy-CFPP correlation model.

18. The machine-readable storage medium of claim 17, wherein the measuring of the vibrational spectrum is part of continuously measuring the vibrational spectrum of hydrocarbon fluid, and wherein the determining of the estimated CFPP is part of monitoring the estimated CFPP over time.

19. The machine-readable storage medium of claim 17, wherein the CFPP is for a total wax concentration.

20. The machine-readable storage medium of claim 17, wherein the CFPP is for a wax concentration at a temperature between about −50° C. to about 20° C.
